(19) United States
(12) Patent Application Publication
(10) Pub. No.: US 2016/0213730 A1
(43) Pub. Date: Jul. 28, 2016

(54) IDENTIFICATION OF NATURAL PLANT EXTRACTS HARBORING ANTI-HEPATITIS C VIRUS NS5B POLYMERASE ACTIVITY

(71) Applicant: Rutgers, The State University of New Jersey, New Brunswick, NJ (US)

(72) Inventors: James E. Simon, Princeton, NJ (US); Qingli Wu, Annandale, NJ (US); Neerja Kaushik-Basu, East Hanover, NJ (US)

(73) Assignee: Rutgers, The State University, New Brunswick, NJ (US)

(21) Appl. No.: 15/088,394

(22) Filed: Apr. 1, 2016

Related U.S. Application Data
(63) Continuation of application No. 13/868,002, filed on Apr. 22, 2013.
(60) Provisional application No. 61/636,254, filed on Apr. 20, 2012.

(51) Int. Cl. A61K 36/79; A61K 45/06; A61K 36/185; A61K 36/074

(52) U.S. Cl. CPC A61K 36/79; A61K 36/074; A61K 45/06; A61K 36/185

(57) ABSTRACT

The present invention relates to a novel use of naturally occurring plants, mushroom, extracts thereof that exhibit properties as HCV NS5B polymerase inhibitors.

### RELATED APPLICATIONS

This application claims priority to the U.S. Provisional Application Ser. No. 61/636,254 filed on Apr. 20, 2012, the content of which is incorporated herein by reference in its entirety.

### FIELD OF INVENTION

The present invention relates to an anti-hepatitis C virus (HCV) composition, in particular relates to a composition having extracts, bioactive fractions and natural compounds extracted or purified from various medicinal plants and a medicinal mushroom, which inhibit HCV replication in cell culture and inhibit HCV NS5B polymerase activity.

### BACKGROUND OF THE INVENTION

Hepatitis C virus (HCV) is a causative agent of persistent liver infections that often progress to chronic hepatitis, cirrhosis, or hepatocellular carcinoma. With an estimated 170-200 million people infected with HCV worldwide, HCV infections are a major public health concern both in developed and developing countries.

HCV, an enveloped virus, is a member of the Flaviviridae family. Similar to other flaviviruses, HCV has a positive-stranded 9.6 kb RNA genome that is translated as a single polyprotein of ˜3000 amino acids. The HCV polyprotein is processed into four structural (core, E1, E2, and p7) and six nonstructural (NS2, NS3, NS4A, NS4B, NS5A, and NS5B) proteins by a combination of cellular and viral proteases, including the HCV serine protease located within NS3. HCV replicates exclusively in the cytoplasm of host cells. Its RNA genome is replicated by the RNA dependent RNA polymerase (RdRp) activity of its 66 kDa nonstructural protein NS5B. Given its essential role in facilitating the replication of the HCV RNA genome, NS5B has emerged as an attractive antiviral target. Additionally, host cells lack RdRp activity, and therefore, NS5B inhibitors are less likely to cause negative effects on host cells than nonspecific broad spectrum viral inhibitors.

Until recently, HCV infections were treated by a combination therapy of pegylated interferon (PEG-IFN) and the nucleoside analog ribavirin. Unfortunately, this therapy was only moderately successful for several reasons, including viral mechanisms to modulate the effects of IFN. The recent clinical approval of HCV-NS3/4A protease inhibitors Victrelis (boceprevir) and Incivek (telaprevir) used in combination with PEG-IFN and ribavirin has substantially improved sustained virological response. While vastly improved, the new anti-HCV drugs are still associated with undesirable effects and cellular toxicity in patients, and the new treatments have complicated dosing regiments, which may limit patient compliance. Further, since HCV exists as quasispecies, the emergence of drug resistant HCV variants during therapy may complicate efforts to clear patients of viremia. Therefore, discovery of novel anti-HCV agents to complement the existing therapies remains a top priority.

### SUMMARY OF THE INVENTION

The present invention relates to a novel use of any one of the naturally occurring plants, mushrooms, plant and mushroom extracts, and specific polyphenols or alkaloids that exhibit properties as HCV NS5B polymerase inhibitors. These are specified as any one or more of the 17 plants and mushrooms described herein and/or their extracts, and six fractionated bioactives from them. In at least one aspect of the invention, each of the extracts exhibits anti-HCV NS5B polymerase activity, functioning as HCV NS5B polymerase inhibitors and thus can be used to treat Hepatitis C. In one embodiment, the present invention relates to methods of inhibiting the NS5B polymerase of HCV using natural plant extracts and their fractionated or purified bioactives, selected from the following non-exhaustive list of extracts obtained from such plants as: Catnip, Gotu Kola, Hibiscus (also called Roselle, Bissap, and includes the green or white, pink, red, dark red types referring to their calyx colors), Holy Basil, Huang Qin, Licorice, Kinkeliba, Kudzu, Moring, Rosemary. In another embodiment the methods of inhibiting the NS5B polymerase of HCV is accomplished by other related extracts obtained from medicinal mushrooms, such as reishi mushroom, or the essential oil of Artemisia annua (also called Sweet Annie, Sweet wormwood) that is obtained through water and/or steam distillation or solvent extraction.

In yet another embodiment, methods for treating HCV hepatitis infection in a mammal are described in patients suffering from such infection or are at risk of developing such infection by administering a therapeutically effective amount of a pharmaceutical composition that contains compounds that would include one or more Anthocyanins; Baicalin; Quercetin; Kaempferol in amounts that is sufficient to treat HCV. In another embodiment, the composition is in the form of a plant extract that contains an alcoholic extract of Catnip, an alcoholic extract of Gotu Kola, an alcoholic extract of Holy Basil, an alcoholic extract of Hibiscus including dark red, green, red, pink and all nonanthocyanin containing varieties (an alcoholic extract of Huang Qin (or skullcap), an alcoholic extract of Kinkeliba, an alcoholic extract of Kudzu, an alcoholic extract of Licorice, an alcoholic extract of Moringa, an alcoholic extract of Purple Basil, an alcoholic extract of Reishi, an alcoholic extract of Rosemary, and an alcoholic extract of Schisandra; and combinations thereof. In yet another embodiment, the anti HCV composition is the aromatic volatile oil or essential oil of Artemisia annua that is obtained through distillation (“Artemisia oil”). The composition can be prepared in pharmaceutically acceptable dosage form such as tablets, capsules, oral suspensions, solutions or emulsions, as well as topical dosage forms including topical creams, emulsions, gels, foams, solutions, or suspensions.

In yet another embodiment, methods for treating HCV hepatitis infection in a mammal are described in patients suffering from such infection or are at risk of developing such infection by administering a therapeutically effective amount of a non-alcoholic composition that contains one or more of the compounds Anthocyanins; Baicalin; Quercetin; Kaempferol in amounts that is sufficient to treat HCV. In at least another embodiment, the composition can be in the form of a non-alcoholic extract, nutritional or dietary drink, herbal tea, chewable gum and the like containing therapeutically effective amounts of Catnip, Gotu Kola, Holy Basil, Hibiscus, Huang Qin, Kinkeliba, Kudzu, Licorice, Moringa, Purple Basil, Reishi, Rosemary, Schisandra; and combinations thereof.

In another aspect of the present invention, the compounds that possess inhibitory properties towards NS5B polymerase of HCV are prepared by the methodologies disclosed herein.

In another aspect of the present invention, kits containing effective therapeutic regimens of interest including additional secondary treatment options for providing combination treatments are envisioned.

### BRIEF DESCRIPTION OF DRAWINGS

FIG. 1 is Table 1 listing all plant species/varieties covered in the present invention.

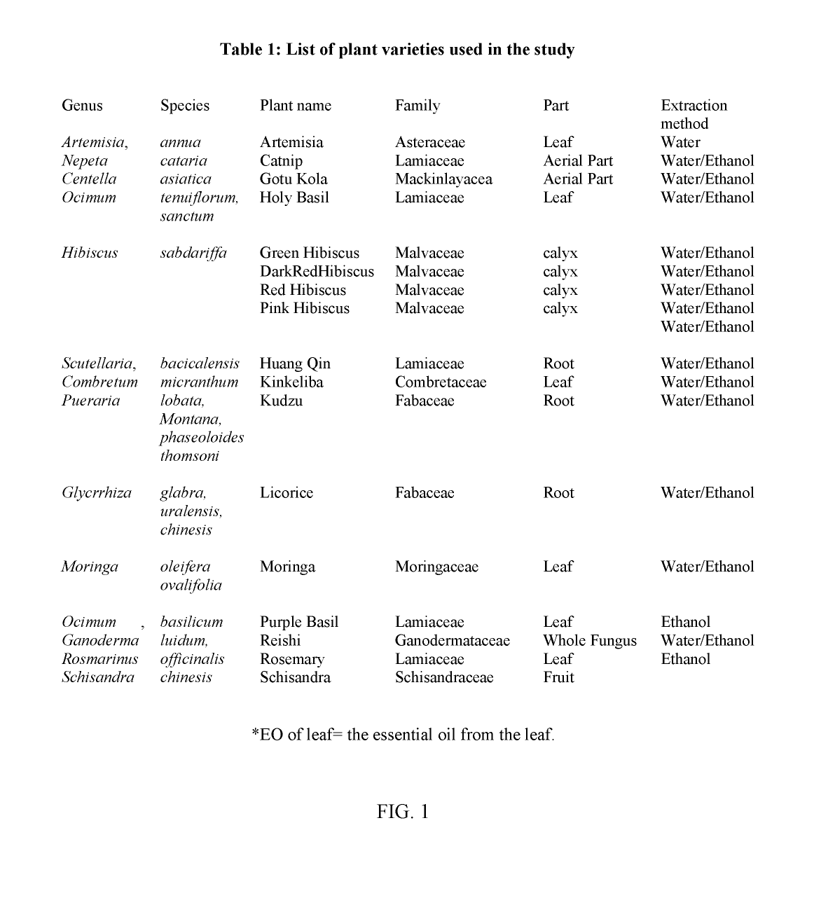

FIG. 2 is Table 2 listing the Anti-HCV effect of plant and mushroom extracts on HCV replicon reporter cells. Huh7/Rep-Feo1b or Huh7.5-FGR-JC1-Rluc2A reporter cells were treated with the indicated plant extract (500μg/mL) for 42 hours. HCV replication was measured by the Firefly luciferase or Renilla luciferase activities of the 1b or 2A replicon reporter cells, respectively. Cell viability was measured by the MTS assay employing the Cell Titer 96AQᵤₑₒᵤₛ One Solution Assay Reagent. Percent inhibition or viability is expressed relative to the DMSO treated controls. Data shown is an average±SD (standard deviation) of at least three independent experiments in duplicate.

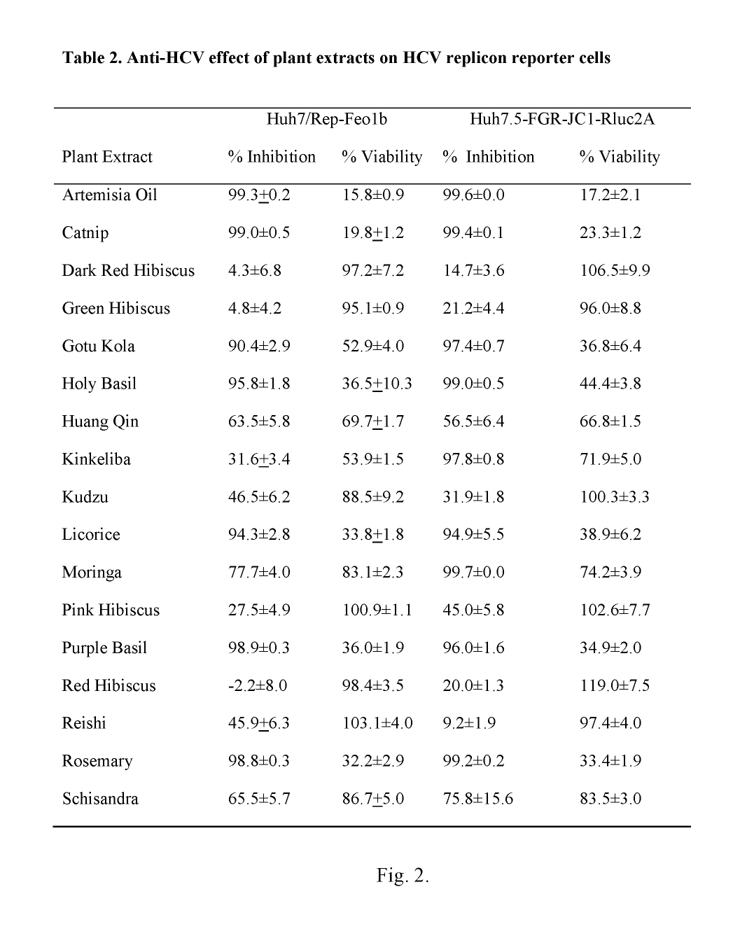

FIG. 3 is Table 3A and 3B. Table 3.A provides the effect of plant and mushroom extracts on intracellular HCV NS5B activity in cell based reporter assay. Table 3.B provides effects of fractionated bioactives on intracellular HCV NS5B activity. BHK-NS5B-FRLuc reporter cells were treated with the indicated plant and mushroom extracts or the indicated fractionated bioactives extract (500 μg/mL) for 42 hours. Cytotoxicity was estimated as the relative levels of Firefly luciferase in compound treated cells versus DMSO controls, while percent inhibition of intracellular NS5B RdRp activity was evaluated from the percent reduction in RLuc to FLuc luminescence signal in extract treated cells versus DMSO controls. The concentration of Quercetin and Kaempferol was 50 μM. Data represents an average±standard deviation of at least three independent experiments in duplicate. (N.d.—Not Determined; n.i.—No Inhibition).

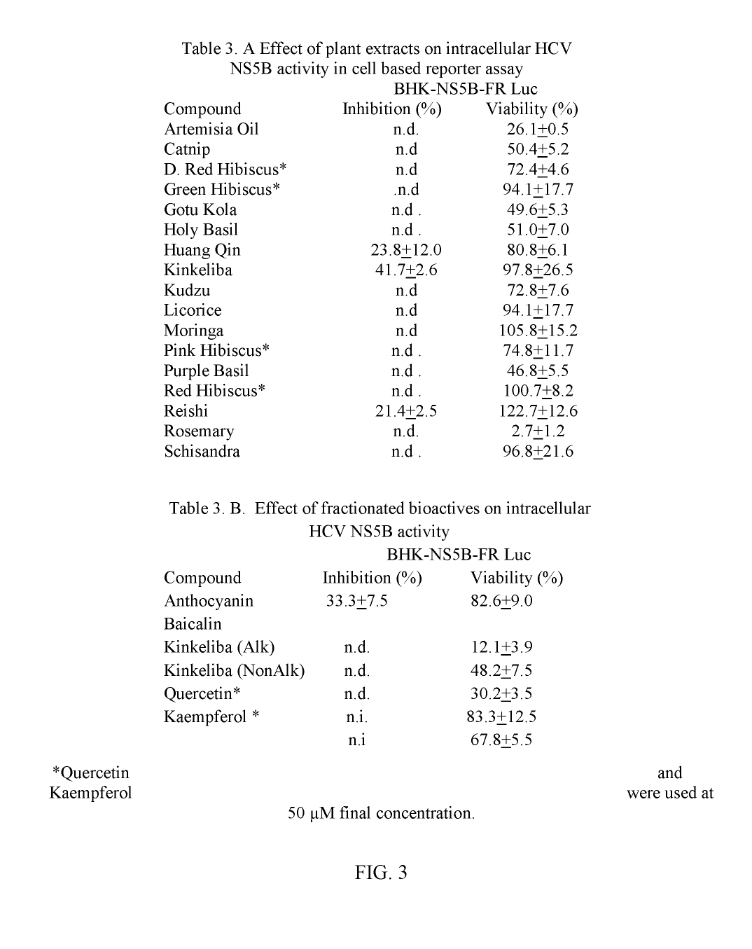

FIG. 4. is Tables 4A and 4B which provide the inhibitory activities of the plant or mushroom extract or the indicated fractionated bioactives on recombinant HCV NS5B. In these Tables the percent inhibition was determined at 100 μg [a] and 50 μg [b] concentrations of the indicated extract in triplicate. The IC₅₀ values [c] of the extracts were determined from dose-response curves employing 8-10 concentrations of each extract in duplicate in two independent experiments. Curves were fitted to data points using nonlinear regression analysis and IC₅₀ values were interpolated using GraphPad Prism 3.03 software. Phenolics [d] were measured using the Folin-Ciocalteau reagent and are expressed as μg chlorogenic acid equivalent (C.A.E.) per milligram wet weight of extract. Values are expressed as an average of at least three independent measurements performed in duplicate.

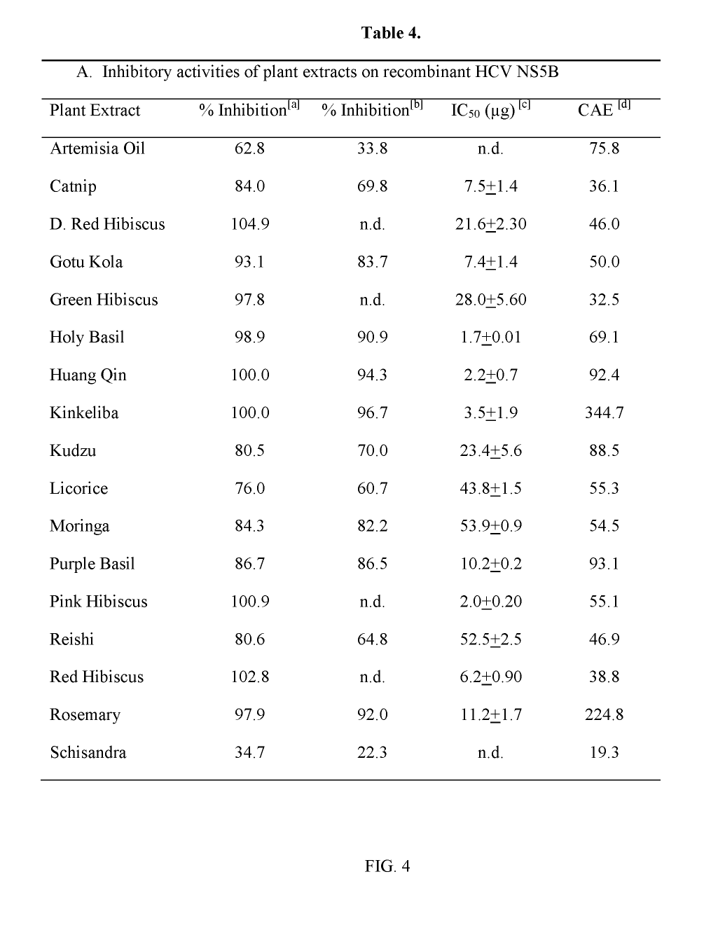

FIG. 5. Table 5 provides the measurements for 50% cytotoxicity (CC₅₀) of the isolated extracts as described in the legends to the table. The antiviral activity of the compounds in Huh7/Rep-Feo1b and Huh7.5-FGR-JC1-Rluc2A was determined at concentrations which had no adverse effect on the cell viability. Similarly the effect of the compounds on intracellular NS5B activity was determined at concentrations which did not affect viability of BHK-NS5B-FRLuc reporter cells. Huh7.5 and BHK-NS5B-FRLuc reporter cells were treated with the indicated extracts at varying concentrations for 48 hours. Cytotoxicity was evaluated by the MTS assay, employing the Cell Titer 96AQᵤₑₒᵤₛ One Solution Assay Reagent inHuh7.5 treated cells, and the relative levels of Firefly luciferase in compound treated cells versus DMSO controls in BHK-NS5B-FRLuc reporter cells.

FIG. 6 is Table 6. Table 6 provides for the effect of Hibiscus extracts in cell based HCV and NS5B reporter assay. Accordingly, Huh7/Rep-Feo1b, Huh7.5-FGR-JC1-Rluc2A and BHK-NS5B-FRLuc reporter cells were treated with the indicated Hibiscus extract at concentration of 500 μg/mL for 48 hours. HCV RNA replication was measured by the Firefly luciferase or Renilla luciferase activities of the 1b or 2A replicon reporter cells, respectively. Percent inhibition is expressed relative to the DMSO treated controls. For BHK-NS5B-FRLuc, percent inhibition of intracellular NS5B RdRp activity was evaluated from the percent reduction in RLuc to FLuc luminescence signal in extract treated cells versus DMSO controls. Data shown is an average±SD (standard deviation) of three independent experiments in duplicate.

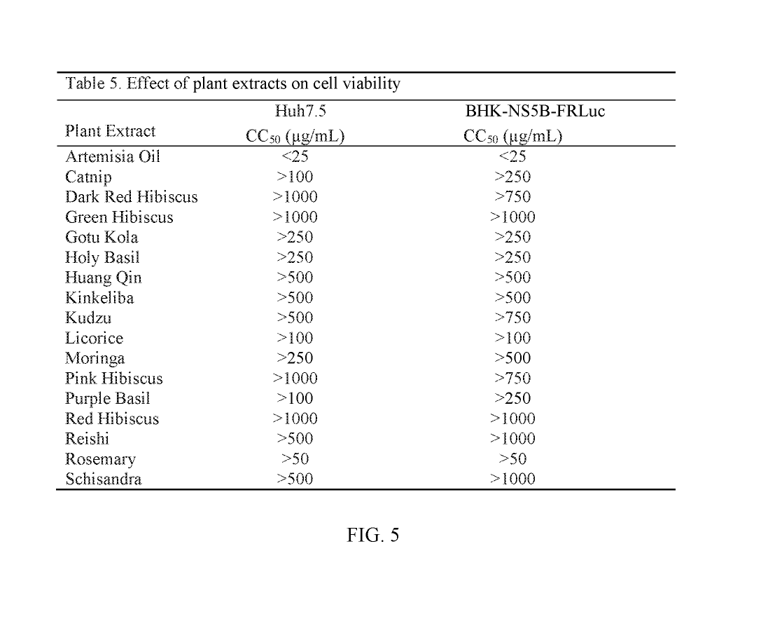

FIG. 7 depicts Table 7. Table 7 provides the effect of the exemplified plant and mushroom extracts in cell based HCV and NS5B reporter assay. Accordingly, Huh7/Rep-Feo1b, Huh7.5-FGR-JC1-Rluc2A and BHK-NS5B-FRLuc reporter cells were treated with the indicated Hibiscus extract at 50 μg/mL concentration for 48 hours. HCV RNA replication was measured by the Firefly luciferase or Renilla luciferase activities of the 1b or 2A replicon reporter cells, respectively. Percent inhibition is expressed relative to the DMSO treated controls. For BHK-NS5B-FRLuc, percent inhibition of intracellular NS5B RdRp activity was evaluated from the percent reduction in RLuc to FLuc luminescence signal in extract treated cells versus DMSO controls. Data shown is an average ± SD (standard deviation) of three independent experiments in duplicate.

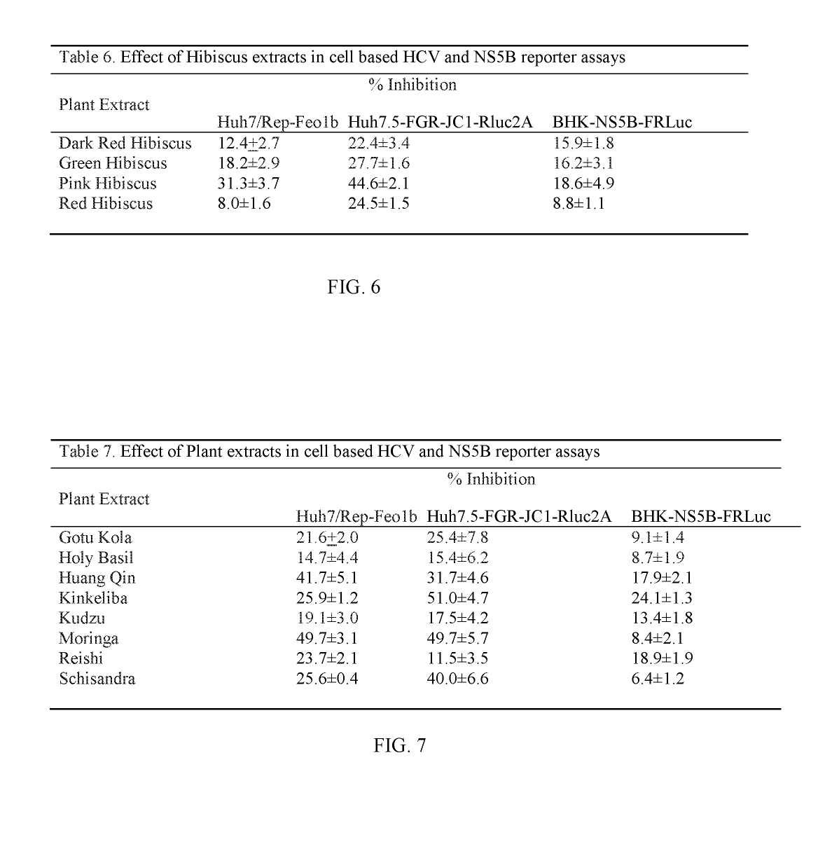

### DETAILED DESCRIPTION OF THE INVENTION AND PREFERRED EMBODIMENTS

The present invention relates to a novel use of naturally occurring plant and mushroom extracts and those with polyphenol properties as HCV NS5B polymerase inhibitors. At least another aspect of the present invention is in part, the unexpected discovery that compositions obtained from selected plants and medicinal mushrooms in the form of extracts, bioactive filtrate or essential oil prepared by such processes as extraction, distillation, and purification, from these plants identified herein are effective in inhibiting the key enzymes of HCV replication. Those of ordinary skill in the art would appreciate that the present inventors have identified components found within natural herbs as having anti-HCV properties in plants and mushrooms generally not recognized in the art to exhibit any anti-hepatitis activity. For example, curcumin, the major component isolated from the curry spice turmeric inhibits the replication of HCV RNA in host cells. Additionally, extracts from Glycyrrhizae radix (Licorice root), Silybum marianum, and Saxifraga melanocentra are reported to block HCV specific processes including RNA replication, viral entry, and polyprotein processing.

Previous research indicates that coumestan—wedelolactone, found in herbal medicines derived from Eclipta prostrata and Wedelia calendulacea, inhibits HCV NS5B activity. Since wedolactone is naturally occurring, there is a strong likelihood anti-NS5B molecules might be present in other plant extracts. The present invention provides other novel HCV NS5B inhibitors found in natural plant and mushroom products that can supplement existing anti-HCV treatments.

In one embodiment, the present invention relates to methods of inhibiting the NS5B polymerase of HCV using natural plant extracts and their fractionated bioactives.

In another embodiment, the present invention is a method of inhibiting the NS5B polymerase of HCV using a composition containing an effective amount of any one of Artemisia Oil (the volatile aromatic or essential oil from Artemisia annua), Anthocyanins, Biacalin, Kaempferol, Quercetin, and the plants and mushrooms or extracts or mushrooms: Catnip (Nepeta cataria and also known as catmint, Nepeta spp.), Hibiscus including dark red, green, red, pink and all nonanthocyanin containing varieties (Hibiscus sabdariffa), Gotu Kola (Centella asiatica), Holy Basil (Ocimum tenuiflorum, Ocimum sanctum), Huang Qin (also called Chinese skullcap, Baical skullcap, skullcap, Scutlellaria, Scutellaria baicalensis, or American skullcap, Scutellaria lateriflora), Kinkeliba (Combretum micranthum), Kudzu (Pueraria spp. including Pueraria lobata, P. Montana, P. edulis, P. phaseoloides and P. thomsoni), Licorice (Glycrrhiza glabra, G. uralensis, G. chinensis), Moringa (Moringa oleifera, M. ovalifolia), Purple Basil (Ocimum basilicum), Reishi (a mushroom, Ganoderma luidum and Ganoderma spp), Rosemary (Rosmarinus officinalis), and Schisandra (Schisandra chinensis).

In a further embodiment, the present invention is a method of inhibiting HCV using plant and mushroom extracts by treating Huh7/huh7.5 replicon cells with plant extracts at a concentration of 500 μg/mL; wherein the extracts are screened for their ability to block HCV RNA replication. Within the scope of the present invention is an extract which contains compounds prepared or identified according to the presently described process steps.

In a more specific embodiment, the present invention is a method of inhibiting the NS5B polymerase of HCV using natural plant or mushroom extracts, wherein such methods follow the steps of determining the effect of the plant or mushroom extracts on cellular viability in Huh7/huh7.5 cells using Aqueous Non-Radioactive Cell Proliferation Assay (MTS) and then administering therapeutically effective amounts of such extracts to subjects in need of HSV treatment.

In another embodiment, the present invention is a method of inhibiting the intracellular NS5B polymerase of HCV by treating BHK-5B-FR Luc cells with plant extracts; wherein these cell lines contain a stably expressed NS5B and a bicstronic reporter gene, (+)FLuc-(−)UTR-RLuc plasmid, which is used to measure cellular viability and NS5B activity respectively. In a further embodiment, the present invention is a method of inhibiting the NS5B polymerase of HCV by testing the effects of plant extracts directly on the NS5B activity via in vitro RNA dependent RNA polymerase (RdRp) assay, and calculating their IC₅₀ values.

In another aspect of the present invention, the compounds of interest having inhibitory effects on the NS5B polymerase of HCV can be in its natural form or in the alternative isolated and purified to be substantially free from naturally associated molecules, ex. at least 75%, 85% or 95% pure as measure by appropriate standard methods such as HPLC analysis.

In yet another aspect of the invention, procedures for obtaining the disclosed extracts are described, wherein such procedures include but is not limited to solvent fractionation using different polar solvent systems, acid-base precipitation, acid-base precipitation with n-butanol extraction and acid-base precipitation with chloroform and n-butanol fractionation.

In one embodiment, the extract of present are prepared by a process that follows the steps of (a) forming a first solution by mixing a plant or a mushroom selected from the group consisting Catnip, Gotu Kola, Holy Basil, Hibiscus, Huang Qin, Kinkeliba, Kudzu, Licorice, Moringa, Purple Basil, Reishi, Rosemary, and Schisandra; and combinations thereof in a solvent selected from the group consisting of an alcohol, such as ethanol; water or a combination thereof (b) forming a second solution by removing the solvent, (c) adjusting the pH to about 9, (d) collecting a fractionated bioactive plant material by continue the steps (a) and (b) to remove sufficient amount of the solvent and optionally (e) suspending the fractionated bioactive plant material in DMSO to a concentration of at least 100 μg/μl.

In at least one embodiment during solvent fractionation, the crude extract may be dissolved in a suitable solvent such as water and partitioned between hexane, chloroform, ethyl acetate, and n-butanol. In at least another embodiment, the alkaloids (in the kinkeliba for example) are focused in an alcohol. In another embodiment, the process further includes a step of acid-base precipitation, wherein the crude extract is dissolved in acetic acid in water and then filtered by vacuum to separate the non-alkaloids that did not dissolve. In another embodiment, the acidity of the solution is brought from pH 3 to pH 9 by the addition of NH₄OH (38% in water) and the solution is allowed to precipitate and settle for 60 min before filtering by vacuuming. In yet another embodiment, the process further contains steps of collecting a bioactive filtrate from the extraction procedure and washing it with distilled water until it ran neutral while the precipitate is dissolved in methanol and dried to obtain the total alkaloid (Alkaloid biofraction) and finally removing the solvent from mother liquid to obtain the Non-Alkaloid biofraction.

In another embodiment, during acid-base precipitation with n-butanol extraction, the crude extract is dissolved in 3% Acetic Acid in water and filtered by vacuum to separate the non-polar components that did not dissolve. The acidic solution will be brought from pH 3 to pH 9 by the addition of NH₄OH (38% in water) and the solution is allowed to precipitate and settle for 60 min before extraction with n-butanol to obtain the total alkaloids (Alkaloid biofraction). In another embodiment, during acid-base precipitation with chloroform and n-butanol fractionation, the crude extract is then dissolved in 3% Acetic Acid in water and filtered by vacuum to separate the non-polar components that did not dissolve. The acidic solution is then brought from pH 3 to pH 9 by the addition of NH₄OH (38% in water) and the solution is allowed to precipitate and settle for 60 min before fractionation with chloroform and n-butanol. The total alkaloids (Alkaloid biofraction) is concentrated and focused in the n-butanol part.

In another embodiment, the extract of the plants or mushrooms obtained are prepared in the form of a medicinal composition. Examples of compositions of the present invention include but are not limited to food, food additives, teas and beverages, nutritional and/or dietary supplements, and pharmaceutical preparations, oral, as well as topical in suitable carriers. Suitable dosage forms may be applied according to acceptable formulation techniques.

Oral formulations suitable for use in the practice of the present invention include capsules, gels, cachets, tablets, effervescent or non-effervescent powders or tablets, powders or granules; as a solution or suspension in aqueous or non-aqueous liquid; or as an oil-in-water liquid emulsion or a water-in-oil emulsion. The formulations for oral administration may comprise a non-toxic, pharmaceutically acceptable, inert carrier such as lactose, starch, sucrose, glucose, methyl cellulose, magnesium stearate, dicalcium phosphate, calcium sulfate, mannitol, sorbitol, cyclodextrin and cyclodextrin derivatives and the like.

Oral dosage forms such as tablets or capsules can be easily formulated and be made easy to swallow or chew. Tablets may contain suitable binders, lubricants, diluents, disintegrating agents, coloring agents, flavoring agents, flow-inducing agents, and melting agents. A tablet may be made by compression or molding, optionally with one or more additional ingredients. Compressed tables may be prepared by compressing the active ingredient in a free flowing form (e.g., powder, granules) optionally mixed with a binder (e.g., gelatin, hydroxypropylmethlcellose), lubricant, inert diluent, preservative, disintegrant (e.g., sodium starch glycolate, cross-linked carboxymethyl cellulose) surface-active or dispersing agent. Suitable binders include starch, gelatin, natural sugars such as glucose or betalactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, xanthan gum, and the like. Molded tables may be made by molding in a suitable machine a mixture of the powdered active ingredient moistened with an inert liquid diluent.

Liquid formulations suitable for oral administration may be in the form of an extract, an emulsion, an aqueous solution or a suspension. The oil phase of the emulsions of the composition used to treat subjects in the present invention may be constituted from known ingredients in a known manner. This phase may contain one or more emulsifiers. For example, the oily phase comprises at least one emulsifier with a fat or an oil or with both a fat and an oil or a hydrophilic emulsifier is included together with a lipophilic emulsifier, which acts as a stabilizer. Together, the emulsifier(s) with or without stabilizer(s) make up an emulsifying wax, and the wax together with the oil and/or fat make up the emulsifying ointment base which forms the oily dispersed phase of the cream formulations. Spray dried and/or dried powdered formulations from any of these plants, their extracts by themselves and/or in combination are also included.

Emulsifiers and emulsion stabilizers suitable for use in the formulation include Tween 60, Span 80, cetosteryl alcohol, myristyl alcohol, glyceryl monostearate and sodium lauryl sulphate, parrafm, straight or branched chain, mono—or dibasic alkyl esters, mineral oil. The choice of suitable oils or fats for the formulation is based on achieving the desired cosmetic properties, the properties required and compatibility with the active ingredient.

The effective therapeutic dose is in such ranges that trigger a therapeutically measureable response up to a maximal dosage in the subject in need of HCV treatment that does not cause undesirable or intolerable side effects. For example, the natural, synthetic or isolated HCV NS5B polymerase inhibitors can be administered in an amount of from about 0.1 μ.l/kg/day to about 100 μ.l/kg/day, and preferably from about 1 μ.l/kg/day to about 20 μ.l/kg/day. These dosage ranges represent quantities of the various components of the composition that are therapeutically effective for treating an active HCV infection or as a preventative or prophylactic measure to reduce the likelihood of infection among patients at risk of developing HCV infection

Such patient population include recipients of blood transfusion, drug abusers particularly IV drug users, heath care workers such as doctors, nurses or laboratory personnel, dialysis patients, family members in contact with a HCV infected patient, high-risk sexually active individuals, recipients of body piercing or tattoos and neonates and new borns to women with HCV. The therapeutic dose of the composition and its respective active compounds can vary depending on the age of the patient, nature and severity of disease, and potency of the composition. In any event, the practitioner is guided by skill and knowledge in the field, and the present invention includes, without limitation, dosages that are effective to achieve the described phenomena. In at least one aspect of the present invention, the composition and the respective active compound is administered for at least 1 week, at least 4 weeks, at least 12 weeks, or at least 24 weeks. In an least another embodiment, methods are described for treating a viral infection or the inflammatory reactions associated or caused by HCV in a patient in need of such treatment by administering to a mammal in need thereof a therapeutically effective amount of a compound such as Anthocyanins; Baicalin; Quercetin; Kaempferol; Artemisia oil in this application refers to the Essential Oil of Artemisia, specifically from Artemisa annua (also known as Sweet Annie, Sweet wormwood, annual wormwood, Qinghao) obtained as the distilled aromatic volatile oil from the leaves, leaves and flowers of the plant Artemisia annua. In at least one embodiment, the Artemisia essential oil is obtained by using fresh, partially dry and/or dry plant materials subjected to water or hydrodistillation, steam distillation, or solvent extraction to procure the aromatic volatiles or essential oils from the plant.

In another embodiment, methods are described for treating a viral infection or the inflammatory reactions associated or caused by HCV in a patient in need of such treatment by administering to a mammal in need thereof a therapeutically effective amount of a compound or a bioactive plant extracts selected from the group consisting of an alcoholic extract of Catnip, an alcoholic extract of Gotu Kola, an alcoholic extract of Holy Basil, an alcoholic extract of Hibiscus, an alcoholic extract of Huang Qin, an alcoholic extract of Kinkeliba, an alcoholic extract of Kudzu, an alcoholic extract of Licorice, an alcoholic extract of Moringa, an alcoholic extract of Purple Basil, an alcoholic extract of the mushroom Reishi, an alcoholic extract of Rosemary, and an alcoholic extract of Schisandra; and combinations thereof.

In an least another embodiment, methods for treating a HCV hepatitis infection in a mammal is described in patients suffering from such infection or are at risk of developing such infection by administering a therapeutically effective amount of a non-alcoholic composition that contains compounds Anthocyanins; Baicalin; Quercetin; Kaempferol in amounts that is sufficient to treat HCV. In at least another embodiment, the composition can be in the form of a non-alcoholic extract, nutritional or dietary drink, herbal tea or chewable gum and the like containing therapeutically effective amounts of Catnip, Gotu Kola, Holy Basil, Hibiscus, Huang Qin, Kinkeliba, Kudzu, Licorice, Moringa, Purple Basil, Reishi, Rosemary, Schisandra; and combinations thereof.

In yet another embodiment, the method include steps of administering an additional therapeutic agent selected from the group consisting of interferons, ribavirin analogs, NS3 protease inhibitors, NS5B polymerase inhibitors, alpha-glucosidase 1 inhibitors, hepatoprotectants, non-nucleoside inhibitors of HCV, and combinations thereof.

In yet another embodiment, a therapeutic kit containing the HCV regimen is disclosed wherein the kit contains (a) a first pharmaceutical composition comprising a compound selected from the group consisting of Anthocyanin; Baicalin; Quercetin; Kaempferol; Artemisia annua essential oil, a plant extract selected from the group consisting of, an alcoholic extract of Catnip, an alcoholic extract of Gotu Kola, an alcoholic extract of Holy Basil, an alcoholic extract of Hibiscus, an alcoholic extract of Huang Qin, an alcoholic extract of Kinkeliba, an alcoholic extract of Kudzu, an alcoholic extract of Licorice, an alcoholic extract of Moringa, an alcoholic extract of Purple Basil, an alcoholic extract of Reishi, an alcoholic extract of Rosemary, and an alcoholic extract of Schisandra; and combinations thereof; and (b) a second pharmaceutical composition comprising an anti-inflammatory agent, and optionally directions for use and optimizing the regimen.

The experiments below are to be construed as merely illustrative and not limitative of the reminder of the disclosure in any way whatsoever. It is believed that those of ordinary skill in the art can based on the description and the examples provided herein appreciate that the inventors of the present invention successfully describe the efficacy of the natural compounds obtained and isolated from specific plants and mushroom in inhibiting HCV NS5B polymerase and subsequently inhibiting the downstream mediators involved in HCV infection or the respective inflammatory reactions.

The flavanoids of the present invention were prepared by methods including, but not limited to, solvent fractionation using different polar solvent systems, acid-base precipitation, acid-base precipitation with n-butanol extraction and acid-base precipitation with chloroform and n-butanol fractionation. During solvent fractionation, the crude extract was dissolved in water and partitioned between hexane, chloroform, ethyl acetate, and n-butanol, along with the remaining water fraction. LC-MS analysis indicated that the alkaloids were focused in the n-butanol fraction. During acid-base precipitation, the crude extract was dissolved in 3% acetic acid in water and filtered by vacuum to separate the non-polar components or non-alkaloids that did not dissolve.

The acidic solution was then brought from pH 3 to pH 9 by the addition of NH4OH (38% in water) and the solution was allowed to precipitate and settle for 60 min before filtering by vacuum. The filtrate was collected and washed with distilled water until it ran neutral while the precipitate was dissolved in methanol and dried to obtain the total alkaloid. During acid-base precipitation with n-butanol extraction, the crude extract was dissolved in 3% Acetic Acid in water and filtered by vacuum to separate the non-polar components that did not dissolve.

The acidic solution was brought from pH 3 to pH 9 by the addition of NH4OH (38% in water) and the solution was allowed to precipitate and settle for 60 min before extraction with n-butanol to obtain the total alkaloids. During acid-base precipitation with chloroform and n-butanol fractionation, the crude extract was then dissolved in 3% Acetic Acid in water and filtered by vacuum to separate the non-polar components that did not dissolve. The acidic solution was brought from pH 3 to pH 9 by the addition of NH4OH (38% in water) and the solution was allowed to precipitate and settle for 60 min before fractionation with chloroform and n-butanol. The total alkaloids were focused in n-butanol part.

### EXPERIMENTAL METHODS

### Preparation of Plant Extracts

Artemisia oil was distilled from dry Artemisia (Artemisia annua) leaf materials. The Reishi (or lingzhi) mushroom (Ganoderma luidum and Ganoderma spp.) and Shisandra (Schisandra chinensis) samples were extracted using ethanol and all the other plant materials using 70% ethanol in water. The powered plant materials were extracted for 3 times. The extraction was combined and the solvent was removed using rotate evaporator to obtain the individuals extracts. Extracts were resuspended in DMSO at a concentration of 100 μg/μl. Each of these plants could be extracted with water and/or different solvents.

### Cell Culture.

BHK-5B-FR-Luc cells were maintained in DMEM media supplemented with 10% fetal calf serum, 10 μg/ml of blasticidin, and 1 mg/ml G418. Huh-7-Rep-Feo1 replicon and Huh7-PFGR-JC1-Rluc2A replicon reporter cells were cultured in DMEM media containing 10% fetal calf serum and 0.5 mg/ml G418 to maintain the HCV sub genomic replicon. All cell lines were cultured at 37° C. in 5% CO₂ atmosphere to a confluence of 80%.

### HCV Replicon Based Luciferase Reporter Assays.

The Huh-7-Rep-Feo1b eplicon was obtained from Naoya Sakamoto and was prepared as described previously by Kim et al 2010. These cell lines contain autonomously replicating HCV sub genomic replicons of genotype 1b and express a Firefly luciferase reporter gene used to measure replication of HCV RNA.

The Huh7.5-FGR-JC1-Rluc2A replicon reporter cells, which contain an autonomously replicating sub genomic HCV strain JFH-1 (genotype 2a) expressing a Renilla luciferase reporter gene were generated in Dr. Hengli Tang's lab and is described herein. Briefly, a plasmid derived from a full length HCV replicon pFGR-JFH-1 was digested with Agel and BsiWI and ligated to a similarly digested p7—plasmid such that the JFH-1 UTR, a neomycin resistance gene, and most of the JFH-1 El was incorporated into the p7Rluc2a plasmid. The resulting 14,806 b.p. construct was linearized by digesting them with Xba I and transcribed in vitro to generate viral RNA. RNA was electroporated into Huh7.5 cells and G418 was used for selection. Cells expressing the HCV proteins and the Renilla luciferase reporter were selected over a period of 3-4 weeks with 0.5 mg/mL G418.

Cells were plated at a density of 1×10⁴ cells per well in a 96-well and subsequently treated with the indicated concentrations of the plant or mushroom extract or the specified fractionated bioactives for periods of 42 or 48 hours as indicated in the Table legend. Following incubation, cells were lysed and luciferase activity was measured using the promega firefly luciferase reporter kit (Promega, USA Cat #E1500) or Renilla luciferase reporter assay (Promega, USA Cat #E2820). Luciferase values were measured with a luminometer. Values are expressed as an average of at least three independent measurements performed in duplicate.

### Cellular Proliferation Assay

To determine the effect of plant extracts on cellular viability, we used the Aqueous Non-Radioactive Cell Proliferation Assay (MTS) from Promega (Cat #G5421). CellTiter 96A_{Queous} One Solution Assay Reagent was used in accordance to the manufacturer's instructions. Values are expressed as an average of at least three independent measurements performed in triplicateⁱ.

### BHK-5B-FR Luciferase Reporter Assays

The BHK-5B-FR Luciferase reporter assay has been previously described. Briefly, BHK-5B-FR Luc cells carry stably expressed NS5B and a bicistronic reporter gene, (+)FLuc-(−)UTR-RLuc, used to measure both cellular viability and NS5B activity. BHK-5B-FR Luc cells were plated at a density of 1×10⁴ cells per well in a 96 well plate. The cells were allowed to incubate for minimum of 8 hours at which point they were treated with the indicated concentrations of the plant or mushroom extract or the specified fractionated bioactives for periods of 42 or 48 hours as indicated in the Table legend. Following the incubation, cellular lysates were harvested and the Promega Dual-Luciferase Reporter Kit (Promega Cat #E1960) was employed to determine firefly and Renilla activity in accordance to the manufacturer's instructions. Luciferase values were recorded using a luminometer. NS5B activity was determined by calculating the ratio of Renilla to firefly luciferase activity. Data represents at least three independent measurements performed in duplicate.

In vitro HCV NS5B RdRp Assay

The RNA dependent RNA polymerase (RdRp) assay used to measure NS5B activity in the presence of inhibitors has been previously described Recombinant NS5B of genotype 1b, which carries an N-terminal histidine-tag, was expressed from the plasmid pTh NS5BCΔ21 in Escherichia coli strain DH5α. NS5B was purified by Ni-NTA chromatography. Initially, reactions were performed in the presence of either 50 or 100 μg of the indicated extract or the equivalent amount of dimethyl sulfoxide (DMSO) in the presence of 250 ng NS5BCΔ21 and 0.25 μM polyrA/U₁₂ template-primer in a reaction buffer containing 20 mM Tris-HCl (pH 7.0), 100 mM NaCl, 100 mM Na-glutamate, 0.1 mM DTT, 0.01% BSA, 0.01% Tween-20, 5% glycerol, 20 U/ml of RNasin, 20 μM UTP, 2 μCi [α-³²P]UTP and 1 mM MnCl₂. Following 90 minute incubation at 30° C., reactions were terminated by the addition of 1 ml of ice-cold 5% trichloroacetic acid (TCA) containing 0.5 mM sodium pyrophosphate. Reactions were precipitated on GF-B filters and washed with TCA to remove unincorporated UTP. Incorporation of radio labeled UTP was measured using liquid scintillation. NS5B activity in the presence of DMSO alone was set at 100% and NS5B activity in the presence of the extract was determined by comparing to NS5B activity in reactions containing DMSO alone. IC₅₀ values of the extracts were determined using 8-10 concentrations in duplicate in a minimal of two independent assays. GraphPad Prism 3.03 software was used to analyze the data. IC₅₀ values were determined using nonlinear regression analysis.

### EXPERIMENTAL RESULTS

### Effects of Plant Extracts on HCV RNA Replication

In order to identify plant extracts with anti-HCV properties, Huh-7-Rep-Feo1b replicon and Huh7.5-FGR-JC1-Rluc2A replicon luciferase reporter cells were employed to rapidly screen for the ability to block replication of HC RNA. The Huh-7-Rep-Feo lbcell line has been described to contain a luciferase reporter gene within an autonomously replicating HCV replicon of 1b. Thus, the system provides a rapid means to identify compounds that block replication of HCV RNA in cell culture. 16 of the 17 extracts screened in this study when applied to cells at a concentration of 500 μg/mL inhibited luciferase activity to at least 30% inhibition when compared to cells treated with DMSO alone (Table 2). While Reishi inhibited luciferase activity ˜46% in the genotype 1b cells, reishi extract had little effect on the replication of the genotype 2a replicon suggesting that the assortment of compounds found in the reishi extract is more specific in inhibiting genotype 1b than 2a. Extracts from 7 of the 17 extracts (Licorice root, Catnip, Gotu Kola, Rosemary, Holy Basil, Purple Basil, and the essential oil of Artemisia annua inhibited replication of both genotypes greater than 90% (Table 2). Replicon cells treated with Moringa also had a reduction in HCV RNA replication ≧77% in both genotypes with inhibition somewhat higher in genotype 2a. Huang Qin and Schisandra extracts treated cells significantly reduced the replication of both replicons with luciferase values inhibited ≧50% (Table 2). 1b replicon was inhibited when treated with Kinkileba (˜32% inhibition) albeit not as robust inhibition as seen with the other extracts (Table 2). However, Kinkeliba extract had a dramatic effect on genotype 2b inhibiting luciferase values in this cell line to 97% suggesting that, like Reishi, compounds within Kinkeliba extract are more effective against a genotype 2a than 1b. Also, there is a slight difference between genotype 1b and 2a replication in Kudzu extract treated cells when comparing luciferase activity of Huh-7-Rep-Feo1 Replicon (˜47% inhibition) to the Huh7.5-PFGR-JC1-Rluc2A replicon (˜32% inhibition) (Table 2). Compared to the other shades of hibiscus, pink hibiscus's ability to inhibit HCV2a replicon replication in cells was remarkably higher. This is the first study that identifies anti-HCV properties contained in these plant extracts, with the exception of licorice extract, which was previously shown to inhibit HCV RNA replication in tissue culture.

### Effect of Plant Extracts on Cellular Viability

MTS assay was used to measure cellular viability in the Huh7/Huh7.5 cells treated with 500μg/mL of extracts. As expected, treatment of the cells with the equivalent amount of DMSO (1% final concentration) had little detectable effect on Huh7 cellular viability. Therefore, viability was set to 100% in DMSO treated cells (Table 2). The effects of all other extracts on cellular viability was compared to viability in DMSO treated cells and measured as a percentage of the DMSO control. Huh7 cells displayed significant cellular toxicity with viability below 50% in cells treated with Licorice, Holy Basil, Catnip, Rosemary, Gotu Kola, Purple Basil, and Artemisia Oil extracts (Table 2). Therefore, there is a possibility that the inhibition of HCV RNA replication by these extracts is due to overall cellular toxicity rather than inhibition of a specific HCV replication process. Alternatively, these extracts may contain compounds that block both the replication of the HCV RNA and induce cellular toxicity. Huang Qin and Kinkeliba were more tolerated with viability well above 50% in both replicon expressing Huh7 lines. The viability of Huh7 cells containing the HCV replicons was virtually unaffected when treated with 500μg/mL of Schisandra, kudzu, Moringa, Hibiscus or Reishi extracts with viability above 80% when compared to the DMSO control (Table 2). Therefore, the inhibition of luciferase activity expressed from the HCV replicon in cells treated with these extracts is most likely due to inhibition of a HCV replication process rather than a general cellular toxicity.

### Effect of Extracts on Intracellular HCV NS5B Activity

To understand the mechanism of the disclosed plant extracts in inhibiting HCV replication the present inventors employed BHK-5B-FR Luc reporter assay. Since many of these compounds inhibited luciferase activity in HCV replicon expressing cells, one possible explanation is that these compounds block NS5B polymerase activity, thus inhibiting replication of the autonomously replicating HCV RNA. BHK-5B-FR Luc reporter assay are used to measure the effects of these extracts on both cellular viability and NS5B activity. This system has been previously described and stably expresses NS5B and a bicistronic reporter of (+)FLuc-(−)UTR-RLuc. Firefly luciferase is used as a measure of cellular viability. Renilla luciferase activity in these cells directly reflects NS5B activity, and the ratio of Renilla luciferase to firefly luciferase values provide a measure of NS5B activity while controlling for any loss in cellular viability. Consistent with data obtained using Huh7 cells, treatment of BHK cells with 500 μg/mL of Catnip, Gotu Kola, Holy Basil, and purple basil resulted in reduced cell viability of ˜50% while the essential oil of Artemisia annua and Rosemary reduced cell viability by greater than 70% at this concentration (Table 3). Licorice extract, which produced dramatic cellular toxicity in Huh7 cells, was not toxic to BHK cells at the same concentration, perhaps reflecting intrinsic differences in sensitivity and permeability in the hamster BHK cells versus the human Huh7 /huh7.5 cells. BHK-5B-FRLuc cells treated with hibiscus extracts, Moringa, Kinkeliba and Schisandra extracts had no apparent effect on cellular viability with firefly luciferase values well above 90% activity when compared to cells treated with DMSO alone (Table 3). Kudzu and Huang Qin extracts were also reasonably tolerated by the BHK cells with viability ≧70%.

BHK-5B-FRLuc cells treated with extracts of Huang Qin, Kinkeliba, and Reishi showed little loss in cellular viability consistent with data obtained using Huh7 cells. But, there was a reduction in the ratio of Renilla to firefly luciferase activity in these extract treated cells when compared to cells treated with DMSO alone indicating that the compounds contained within these extracts reduced intracellular NS5B activity. Reishi and Huang Qin modestly inhibited NS5B activity at ˜20%. However, Kinkeliba dramatically affected activity of the viral polymerase with ˜40% inhibition of NS5B activity in BHK-NS5B cells treated (Table 3). Therefore, the limited effect on cellular viability combined with the anti-NS5B activity and inhibition of HCV replicons in cell culture make Kinkeliba, Reishi, and Huang Qin extracts a potential source of HCV inhibitory compounds that may function as scaffolds for the development of future NS5B inhibitors. Other extracts that inhibit intracellular NS5B RdRp activity but do not effect the cellular viability will also be useful as anti-hepatitis agents.

RNA dependent RNA polymerase (RdRp) assay was employed to determine if the extracts could block NS5B mediated incorporation of a radiolabeled UTP into a PolyA/U₁₂ template. This assay has been previous described and used successfully to identify NS5B inhibitors (Kaushik-Basu et al., 2008a; Kaushik-Basu et al., 2008b; Musmuca et al., 2010; Rawal et al., 2008; Talele et al., 2010). Further, the in vino RdRp assay tests extracts for the ability to directly inhibit NS5B without the need to permeate cellular membranes. With the exceptions of Schisandra, the essential oil of Artemisia annua, and Licorice extracts, at 100 μg per reaction, all extracts tested inhibited recombinant NS5B activity at greater than 80% inhibition compared to NS5B reactions in the presence of DMSO alone (Table 4). Further, NS5B RdRp activity was potently inhibited ≧90% in the presence of just 50 μg of either Holy Basil, Huang Qin, Kinkeliba, or Rosemary extract (Table 4). At 100 μg per reaction, all Hibiscus extracts blocked recombinant NS5B-mediated incorporation of a radiolabeled UTP into a PolyA/U₁₂ RNA template at greater than 97% inhibition when compared to reactions containing DMSO alone. Gotu kola, Moringa, and purple basil extract at 50 μg per reaction also inhibited NS5B activity ≧80%. Reactions containing 50 μg of Catnip, Kudzu, Licorice, and Reishi extract inhibited NS5B activity ˜60-70%. At 50 μg, the essential oil of Artemisia annua and Schisandra extract had little effect on NS5B activity with only ˜20-30% inhibition ruling out the possibility that plant extracts nonspecifically block HCV NS5B (Table 4). Schisandra was such a poor inhibitor of NS5B activity in the RdRp assay despite its potency as an HCV replicon inhibitor. In that respect, Schisandra may have additional targets other than NS5B that inhibit HCV RNA replication in the replicon system.

IC₅₀ values of the compounds indicate extracts from Licorice, Kudzu, and Moringa Oleifera were moderate inhibitors of NS5B activity with IC₅₀ values between ˜44 and 54 μg per reaction (Table 4). Kudzu extract functioned with a slightly lower IC₅₀ of ˜24 μg per reaction (Table 4). Holy basil, Huang Qin, Catnip, Gotu Kola, Rosemary, Purple Basil, Kinkeliba, and Reishi extracts were potent inhibitors of NS5B activity with IC₅₀ values well below 12 μg per reaction (Table 4), making the compounds found within these extracts an excellent source of HCV NS5B inhibitors. Green Hibiscus and dark Red Hibiscus had IC₅₀ values of ˜28 and 22 μg per reaction respectively. Red Hibiscus and pink Hibiscus were even more potent inhibitors of NS5B activity with IC₅₀ values well below 10 μg per reaction. Because Schisandra and the essential oil of Artemisia annua did not robustly inhibit NS5B activity at either 50 μg or 100 μg per reaction (Table 4), the IC₅₀ values for these extracts were not determined.

### Measure of Polyphenol Content in Plant Extracts

Because many compounds found in natural products that have antiviral and specifically anti-HCV properties are known to be polyphenol, the polyphenol content of extracts were determined based on a linear standard curve of chlorogenic acid. Additionally, knowing the polyphenol content in the extract provides a means to normalize future extracts. Interestingly, Schisandra extract, which had the lowest concentration of polyphenol compounds (19 μg/mg extract), had the lowest amount of inhibition against the RdRp activity of recombinant NS5B when compared to the other extracts suggesting that the amount of polyphenolic compounds in extracts is important in inhibiting NS5B activity (Table 4). In contrast, kinkeliba, which was a potent inhibitor of in vitro NS5B activity and NS5B activity within the BHK reporter cells had the highest concentration of polyphenol content (345 μg/mg extract) (Table 4). Rosemary extract also had a high concentration of polyphenol content that corresponded to a high inhibition of in vitro NS5B activity (Table 4). All other extracts had similar polyphenolic content (Table 4). Based on such finding polyphenols with similar characteristics would be useful as against HCV infection.

For example, Hibiscus polyphenols include the two well characterized anthocynidins, delphinidin-3-sambubioside and cyanidin-3-sambubioside, the structures of which is broadly depicted as wherein R is OH, or H.

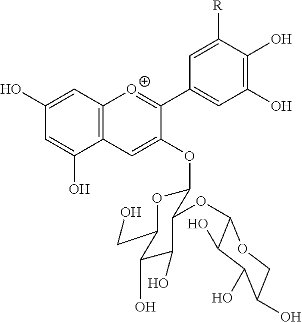

Those of ordinary skill in the art would appreciate the fact that such Hibscius polyphenols once isolated individually or in combination with others can enhance the anti-viral activity of against HCV.

Similarly, the leaves of Moringa collected from sub-Sahara Africa when analyzed shows to be rich in the content of phenolic components by HPLC-UV-MS. Twelve flavonoids identified, include quercetin and kaempferol glucosides and glucosidemalonates as major constituents. To facilitate quantitative analysis, acid hydrolysis during extraction of moringa samples were employed to convert the conjugates into their respective flavonoid aglycones, allowing accurate quantitation of total flavonoids as aglycones.

### Effect of Plant Extract on Cell Viability

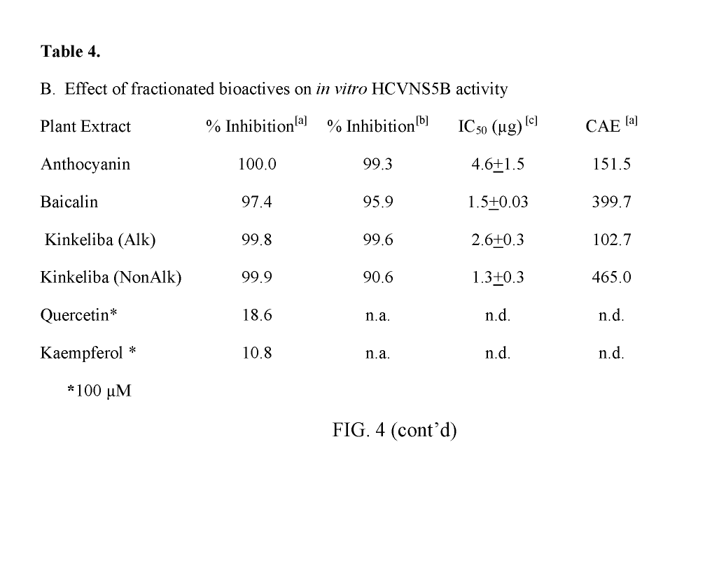

The present inventors have also determined the antiviral activity of the compounds of the present invention in Huh7/Rep-Feo1 and Huh7.5-FGR-JC1-Rluc2A at concentrations which had no adverse effect on the cell viability. (See FIG. 5). The CC50 of the compounds evaluated showed that the antiviral activity of the compounds in Huh7.5-FGR-JCL-Rlue2A had no adverse effect on the cell viability. Huh7.5 and BHK-NS5B-FRLuc reporter cells were treated with the indicated extracts at varying concentrations for 48 hours. Cytotoxicity was evaluated by the MTS assay, employing the Cell Titer 96AQᵤₑₒᵤₛ One Solution Assay Reagent in Huh7.5 treated cells, and the relative levels of Firefly luciferase in compound treated cells versus DMSO controls in BHK-NS5B-FRLuc reporter cells. (FIG. 5).

Those of ordinary skill in the art can appreciate that each one of such polyphenols alone or in combination can improve the clinical therapeutic efficacy of the presently disclosed anti-HCV treatments. While the invention has been described with references to specific embodiments, modifications and variations of the invention may be construed without departing from the scope of the invention, which is defined in the following claims.

1-22. (canceled)

23. A therapeutic extract prepared by a process comprising: (a) forming a first solution by mixing a plant selected from the group consisting of Catnip, Gotu Kola, Holy Basil, Hibiscus, Huang Qin, Kudzu, Licorice, Moringa, Purple Basil, Reishi, Rosemary, Schisandra, and combinations thereof in alcohol or an alcohol-water mixture; (b) forming a second solution by removing the solvent; (c) adjusting the pH to about 9; (d) collecting a fractionated bioactive plant material by continuing the steps (a) and (b) to remove a sufficient amount of the solvent; and (e) suspending the fractionated bioactive plant material in DMSO to a concentration of at least 100 μg/μl.

24. The extract of claim 23, further comprising a compound selected from the group consisting of Anthocyanins, Baicalin, Quercetin, and Kaempferol.

25. The extract of claim 23, wherein the weight ratio of the fractionated bioactive plant material to DMSO is 10:0.1 to 0.1:10.

26. The extract of claim 23, further undergoing a purification, filtration, extraction process.

27. A therapeutic composition consisting essentially of the extract of claim 23, a carrier, and optionally a secondary active ingredient selected from the group consisting of interferons, ribavirin analogs, NS3 protease inhibitors, NS5B polymerase inhibitors, alpha-glucosidase 1 inhibitors, hepatoprotectants, non-nucleoside inhibitors of HCV, and combinations thereof.

28. A therapeutic composition comprising extract of the Hibiscus and an organic solvent and a pharmaceutically acceptable carrier, wherein the extract of Hibiscus is in a sufficient amount to inhibit NS5B polymerase activity of the hepatitis C virus (HCV), and wherein the extract of Hibiscus is prepared by a process comprising: (a) forming a first solution by mixing Hibiscus in alcohol or an alcohol-water mixture; (b) forming a second solution by removing the solvent; (c) adjusting the pH to about 9; and (d) collecting a fractionated bioactive plant material by continuing the steps (a) and (b) to remove a sufficient amount of the solvent.

29. The therapeutic composition of claim 28, wherein the composition has an IC₅₀ value of below 10 μg.

30. The therapeutic composition of claim 28, wherein the Hibiscus is selected from the group consisting of dark red, green, red, pink and all nonanthocyanin containing varieties (Hibiscus sabdariffa).
